(19) United States
(12) Patent Application Publication
(10) Pub. No.: US 2025/0153181 A1
(43) Pub. Date: May 15, 2025

(54) MICROFLUIDICS DEVICE INCLUDING GANGED RESERVOIR CONFIGURATIONS AND METHODS OF USING SAME

(71) Applicant: Nicoya Lifesciences Inc., Kitchener (CA)

(72) Inventors: Sebastian VON DER ECKEN, Kitchener (CA); Ian SWYER, Kitchener (CA)

(21) Appl. No.: 18/839,352

(22) PCT Filed: Sep. 8, 2023
(86) PCT No.: PCT/CA2023/051186

§ 371 Date: Aug. 16, 2024

Related U.S. Application Data
(60) Provisional application No. 63/405,207, filed on Sep. 9, 2022.

(51) Int. Cl. B01L 3/00

(52) U.S. Cl. CPC B01L 3/502792; B01L 2200/0605; B01L 2200/16; B01L 2300/0816; B01L 2300/0851; B01L 2300/0887; B01L 2300/165; B01L 2400/0427

(57) ABSTRACT

Described herein are devices and methods for fluid manipulation using electrowetting. More specifically, this disclosure provides devices and methods for manipulating fluids using a digital microfluidics (“DMF”) device. For example, a fluid may be provided to a reservoir of a DMF device. The reservoir of the DMF device may include an electrode array configured to discretize the fluid into more than one large-volume droplets, each of which may be individually manipulated within the reservoir. Any one of the more than one large-volume droplets may be primed or split into one or more medium-volume droplets. The primed droplets or medium-volume droplets may further be split into one or more small-volume droplets for further processing on the DMF device. Accordingly, the described devices and methods enable high throughput and space savings on the DMF device.

### CROSS-REFERENCE

This application claims the benefit of U.S. Provisional Application No. 63/405,207, filed Sep. 9, 2022, which is hereby incorporated by reference in its entirety herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### FIELD OF THE INVENTION

The subject matter relates generally to systems and devices for processing biological materials and more particularly to a microfluidics device including ganged reservoir configurations and methods of using same.

### BACKGROUND

Microfluidics systems and devices are used in a variety of applications to manipulate, process and/or analyze biological materials. Examples of microfluidics devices include droplet actuators, microfluidics cartridges, digital microfluidics (DMF) devices, DMF cartridges, droplet actuators, flow cell devices, and the like. Microfluidics devices generally include two substrates arranged with a gap therebetween. In DMF applications, electrodes are associated with the substrates and arranged to conduct droplet operations via electrowetting.

Certain drawbacks exist with respect to microfluidics devices. For example, as time goes on, there is greater demand to process more and more samples on a single microfluidics device. Accordingly, as the number of samples increases, there is need to increase proportionally the amount of other liquids needed to support the biological processes. Unfortunately, the amount of real-estate on a microfluidics device is limited and adding wells (or reservoirs) for meeting these additional requirements for liquids is challenging. Accordingly, there is a need for improved systems and devices to accommodate high sample throughput.

### SUMMARY

In some aspects, the present disclosure provides a microfluidic device including: a top substrate; a bottom substrate; a gap between the top substrate and the bottom substrate; and an electrode array disposed near the gap for performing droplet operations within the gap. In some embodiments, the electrode array may include one or more arrays of reservoir electrodes, each of the one or more arrays of reservoir electrodes configured to individually manipulate more than one large-volume droplets within the gap. In some embodiments, the electrode array may include one or more arrays of priming electrodes for priming at least one of the more than one large-volume droplets. In some embodiments, the electrode array may include one or more arrays of dispensing electrodes for dispensing into the gap a small-volume droplet from a primed large-volume droplet.

In some embodiments, priming at least one of the more than one large-volume droplets includes forming one or more medium-volume droplets.

In some embodiments, dispensing into the gap includes separating the small-volume droplet from any one of the one or more medium-volume droplets.

In some embodiments, the one or more arrays of reservoir electrodes and the one or more arrays of priming electrodes are the same.

In some embodiments, the electrode array further includes more than one array of reservoir electrodes.

In some embodiments, the electrode array further includes one or more arrays of bridging electrodes between each of the more than one array of reservoir electrodes for manipulating any one of the more than one large-volume droplets between each of the more than one array of reservoir electrodes.

In some embodiments, the electrode array includes at least one of the one or more arrays of reservoir electrodes is in a linear configuration.

In some embodiments, the electrode array includes at least one of the one or more arrays of reservoir electrodes is in a loop configuration.

In some embodiments, a gap height of the gap is from about 50 μm to about 3000 μm.

In some embodiments, a gap height of the gap is greater near the one or more arrays of reservoir electrodes when compared with the gap height of the gap near the one or more arrays of dispensing electrodes.

In some embodiments, the gap height of the gap near the one or more arrays of reservoir electrodes is from about 300 μm to about 3000 μm.

In some embodiments, the gap height of the gap near the one or more arrays of dispensing electrodes is from about 50 μm to about 500 μm.

In some embodiments, the one or more large-volume droplets has a volume from about 20 μL to about 5000 μL.

In some embodiments, the small-volume droplet has a volume from about 1 μL to about 10 μL.

In some embodiments, the top plate includes one or more loading ports for providing the more than one large-volume droplets.

In some embodiments, the one or more loading ports is configured to interface with a micropipette.

In some embodiments, the electrode array further includes an array of processing electrodes for manipulating the small-volume droplet.

In some embodiments, manipulating the small-volume droplet includes either i) mixing the small-volume droplet with one or more additional droplets; ii) splitting the small-volume droplet into one or more additional droplets; or iii) sensing one or more reagents, analytes, or samples of the small-volume droplet.

In some embodiments, the electrode array further includes an array of waste electrodes for disposing of an unused portion of any one of the more than one large-volume droplets.

In some embodiments, the electrode array further includes an array of mixing electrodes for mixing two or more large-volume droplets.

In some embodiments, at least one of the top plate or bottom plate further includes a boundary feature to prevent migration of the more than one large-volume droplets.

In some embodiments, the boundary feature includes a protrusion disposed on either the top substrate or bottom substrate.

In some embodiments, the microfluidic device is a microfluidic cartridge.

In some aspects, the present disclosure provides a method for fluid manipulation. The method includes the steps of providing a microfluidic device, separating a fluid into more than one large-volume droplets, priming one of the more than one large-volume droplets, and dispensing into the gap a small-volume droplet from a primed large-volume droplet.

In some embodiments, the microfluidic device may include a top substrate, a bottom substrate, a gap between the top substrate and the bottom substrate, and an electrode array disposed near the gap for performing droplet operations within the gap.

In some embodiments, the electrode array may include one or more arrays of reservoir electrodes. In some embodiments, each of the one or more arrays of reservoir electrodes is configured to individually manipulate more than one large-volume droplets within the gap. In some embodiments, the electrode array may include one or more arrays of priming electrodes for priming at least one of the more than one large-volume droplets. In some embodiments, the electrode array may include one or more arrays of dispensing electrodes for dispensing into the gap a small-volume droplet from a primed large-volume droplet; loading a fluid onto the microfluidic device.

In some embodiments, priming at least one of the more than one large-volume droplets includes forming one or more medium-volume droplets.

In some embodiments, dispensing into the gap includes separating the small-volume droplet from any one of the one or more medium-volume droplets.

In some embodiments, the one or more arrays of reservoir electrodes and the one or more arrays of priming electrodes are the same.

In some embodiments, the electrode array further includes more than one array of reservoir electrodes.

In some embodiments, the electrode array further includes one or more arrays of bridging electrodes between each of the more than one array of reservoir electrodes for manipulating any one of the more than one large-volume droplets between each of the more than one array of reservoir electrodes.

In some embodiments, at least one of the one or more arrays of reservoir electrodes is in a linear configuration.

In some embodiments, at least one of the one or more arrays of reservoir electrodes is in a loop configuration.

In some embodiments, a gap height of the gap is from about 50 μm to about 3000 μm.

In some embodiments, a gap height of the gap is greater near the one or more arrays of reservoir electrodes when compared with the gap height of the gap near the one or more arrays of dispensing electrodes.

In some embodiments, the gap height of the gap near the one or more arrays of reservoir electrodes is from about 300 μm to about 3000 μm.

In some embodiments, the gap height of the gap near the one or more arrays of dispensing electrodes is from about 50 μm to about 500 μm.

In some embodiments, the one or more large-volume droplets has a volume from about 20 μL to about 5000 μL.

In some embodiments, the small-volume droplet has a volume from about 1 μL to about 10 μL.

In some embodiments, the top plate includes one or more loading ports for providing the more than one large-volume droplets.

In some embodiments, the one or more loading ports is configured to interface with a micropipette.

In some embodiments, the electrode array further includes an array of processing electrodes for manipulating the small-volume droplet.

In some embodiments, manipulating the small-volume droplet includes either i) mixing the small-volume droplet with one or more additional droplets; ii) splitting the small-volume droplet into one or more additional droplets; or iii) sensing one or more reagents, analytes, or samples of the small-volume droplet.

In some embodiments, the electrode array further includes an array of waste electrodes for disposing of an unused portion of any one of the more than one large-volume droplets.

In some embodiments, the electrode array further includes an array of mixing electrodes for mixing two or more large-volume droplets.

In some embodiments, at least one of the top plate or bottom plate further includes a boundary feature to prevent migration of the more than one large-volume droplets.

In some embodiments, the boundary feature includes a protrusion disposed on either the top substrate or bottom substrate.

In some embodiments, the microfluidic device is a microfluidic cartridge.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 and FIG. 2 illustrate plan views and cross-sectional views of an example of a linear ganged reservoir configuration including multiple storage locations and multiple loading ports, in accordance with an embodiment of the disclosure;

FIG. 3 and FIG. 4 illustrate plan views and cross-sectional views of an example of a linear ganged reservoir configuration including four storage locations and four loading ports, in accordance with an embodiment of the disclosure;

FIG. 5 and FIG. 6 illustrate plan views and cross-sectional views of an example of a linear ganged reservoir configuration including multiple storage locations and one loading port, in accordance with an embodiment of the disclosure;

FIG. 7 and FIG. 8 illustrate plan views and cross-sectional views of an example of a linear ganged reservoir configuration including four storage locations and one loading port, in accordance with an embodiment of the disclosure;

FIG. 9A illustrates a cross-sectional view of an example of a conventional well or reservoir;

FIG. 9B and FIG. 9C illustrate plan views of example configurations of conventional wells or reservoirs;

FIG. 9D illustrates a plan view of an example of a linear ganged reservoir and showing the potential real-estate or space savings compared with the conventional well configurations shown in FIG. 9B and FIG. 9C;

FIG. 10 illustrates a plan view of an example of a ganged reservoir configuration including multiple linear ganged reservoirs arranged side-by-side, in accordance with an embodiment of the disclosure;

FIG. 11 illustrates a plan view of an example of a 4×4-ganged reservoir including four ×4-linear ganged reservoirs arranged side-by-side, which is one example of the ganged reservoir configuration shown in FIG. 10;

FIG. 12 illustrates a plan view of an example of a ganged-reservoir array configuration including bridge electrodes for transporting liquids between lanes of linear ganged reservoirs and including multiple outlet storage locations, in accordance with an embodiment of the disclosure;

FIG. 13 illustrates a plan view of an example of a ganged-reservoir array configuration including bridge electrodes for transporting liquids between lanes of storage locations and including one outlet storage location, in accordance with an embodiment of the disclosure;

FIG. 14 and FIG. 15 illustrate plan views of an example of a 5×5 ganged-reservoir array including one outlet storage location, in accordance with an embodiment of the disclosure;

FIG. 16, FIG. 17a, and FIG. 17b illustrate plan views of examples of ganged-reservoir array configurations including multiple bridge electrodes and/or different shaped bridge electrodes, in accordance with an embodiment of the disclosure;

FIG. 18 and FIG. 19 illustrate plan views of an example of a looped ganged reservoir, in accordance with an embodiment of the disclosure;

FIG. 20 illustrates a plan view of an example of a cascading linear ganged reservoir including different sized storage locations arranged from small-volume to large-volume, in accordance with an embodiment of the disclosure;

FIG. 21 and FIG. 22 illustrate plan views of examples of electrode arrangements including linear ganged reservoirs in relation to a processing area and a waste collection area, in accordance with an embodiment of the disclosure;

FIG. 23 illustrates a plan view of example of electrode arrangements including a linear ganged reservoir and looped ganged reservoir in relation to a mixing area, processing area and a waste collection area, in accordance with an embodiment of the disclosure;

FIG. 24 illustrates a plan view of an example of a microfluidics device including the ganged reservoir configurations; in accordance with an embodiment of the disclosure;

FIG. 25 illustrates a plan view and a cross-sectional view of a linear ganged (or extended) reservoir configuration that further includes a boundary feature, in accordance with an embodiment of the disclosure; and

FIG. 26 illustrates a flow diagram of a method of using the ganged reservoirs, in accordance with an embodiment of the disclosure.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

In some embodiments, the subject matter provides a microfluidics device including ganged (or extended) reservoir configurations and methods of using same.

In some embodiments, the ganged (or extended) reservoir configurations and methods may provide a dispenser portion, a primer portion, and a reservoir portion and wherein the reservoir portion may include any arrangement of two or more liquid storage locations.

In some embodiments, the ganged (or extended) reservoir configurations and methods may be provided wherein a reservoir portion including two or more liquid storage locations supplies liquid to a primer portion and wherein the primer portion supplies liquid to a dispenser portion.

In some embodiments, the ganged (or extended) reservoir configurations and methods may provide a reservoir portion including two or more liquid storage locations and wherein the liquid storage locations may be arranged linearly (i.e., one-dimensionally (1D)).

In some embodiments, the ganged (or extended) reservoir configurations and methods may provide a reservoir portion including two or more liquid storage locations and wherein the liquid storage locations may be arranged in an array (i.e., two-dimensionally (2D)).

In some embodiments, the ganged (or extended) reservoir configurations and methods may provide a reservoir portion including two or more liquid storage locations and wherein the liquid storage locations may be arranged in a loop (i.e., 2D).

In some embodiments, the ganged (or extended) reservoir configurations and methods may provide a reservoir portion including any arrangement of two or more liquid storage locations for holding and managing large-volume droplets and wherein the large-volume droplets may be the same type of droplets or different types of droplets.

In some embodiments, the ganged (or extended) reservoir configurations and methods may provide a reservoir portion including any arrangement of two or more liquid storage locations for dispensing mid-volume droplets to a primer portion.

In some embodiments, the ganged (or extended) reservoir configurations and methods may provide a primer portion for dispensing small-volume droplets to a dispenser portion.

In some embodiments, the ganged (or extended) reservoir configurations and methods may provide a dispenser portion for dispensing small-volume droplets to the processing area of a microfluidics device.

In some embodiments, the ganged (or extended) reservoir configurations and methods may provide a process by which multiple large-volume droplets at multiple storage locations may be used to resupply mid-volume droplets to one primer portion and then resupply small-volume droplets to one dispenser portion.

### Terms and Definitions

Unless otherwise defined, all technical terms used herein have the same meaning as commonly understood by one of ordinary skill in the art to which this disclosure belongs.

The term “activate,” with reference to one or more electrodes, means affecting a change in the electrical state of the one or more electrodes which, in the presence of a droplet, results in a droplet operation. Activation of an electrode can be accomplished using alternating current (AC) or direct current (DC). Any suitable voltage may be used. For example, an electrode may be activated using a voltage which is greater than about 5 V, or greater than about 20 V, or greater than about 40 V, or greater than about 100 V, or greater than about 200 V or greater than about 300 V. Further, electrode may be activated using a positive and/or negative voltage relative to system ground. Further, deactivated electrodes may be held at ground or floated. The suitable voltage being a function of the dielectric's properties such as thickness and dielectric constant, liquid properties such as viscosity and many other factors as well. Where an AC signal is used, any suitable frequency may be employed. For example, an electrode may be activated using an AC signal having a frequency from about 1 Hz to about 10 MHz, or from about 1 Hz and 10 KHz, or from about 10 Hz to about 240 Hz, or about 60 Hz.

The term “droplet” means a volume of liquid on a droplet actuator. Typically, a droplet is at least partially bounded by a filler fluid. For example, a droplet may be completely surrounded by a filler fluid or may be bounded by filler fluid and one or more surfaces of the droplet actuator. As another example, a droplet may be bounded by filler fluid, one or more surfaces of the droplet actuator, and/or the atmosphere. As yet another example, a droplet may be bounded by filler fluid and the atmosphere. Droplets may, for example, be aqueous or non-aqueous or may be mixtures or emulsions including aqueous and non-aqueous components.

The term “droplet actuator” means a device for manipulating droplets. Microfluidics devices, microfluidics cartridges, digital microfluidics (DMF) devices, and DMF cartridges are examples of droplet actuators. Certain droplet actuators will include one or more substrates arranged with a droplet operations gap therebetween and electrodes associated with (e.g., patterned on, layered on, attached to, and/or embedded in) the one or more substrates and arranged to conduct one or more droplet operations. For example, certain droplet actuators will include a base (or bottom) substrate, droplet operations electrodes associated with the substrate, one or more dielectric layers atop the substrate and/or electrodes, and optionally one or more hydrophobic layers atop the substrate, dielectric layers and/or the electrodes forming a droplet operations surface. A top substrate may also be provided, which is separated from the droplet operations surface by a gap, commonly referred to as a droplet operations gap. Droplet actuators will include various electrode arrangements on the bottom and/or top substrates. During droplet operations it is preferred that droplets remain in continuous contact or frequent contact with a ground or reference electrode. A ground or reference electrode may be associated with the top substrate facing the gap, the bottom substrate facing the gap, or within the gap itself. Where electrodes are provided on both substrates, electrical contacts for coupling the electrodes to a droplet actuator instrument for controlling or monitoring the electrodes may be associated with one or both plates. In some cases, electrodes on one substrate are electrically coupled to the other substrate so that only one substrate is in contact with the droplet actuator. Where multiple substrates are used, a spacer may be provided between the substrates to determine the height of the gap therebetween and define on-actuator dispensing reservoirs. The spacer height may, for example, be from about 5 μm to about 1000 μm, or about 100 μm to about 400 μm, or about 200 μm to about 350 μm, or about 250 μm to about 300 μm, or about 275 μm. The spacer may, for example, be formed of features or layers projecting from the top or bottom substrates, and/or a material inserted between the top and bottom substrates. One or more openings may be provided in the one or more substrates for forming a fluid path through which liquid may be delivered into the droplet operations gap.

In some cases, the top and/or bottom substrate of a droplet actuator includes a PCB substrate that is coated with a dielectric, such as a polyimide dielectric, which may in some cases also be coated or otherwise treated to make the droplet operations surface hydrophobic. Various materials are also suitable for use as the dielectric component of the droplet actuator. In some cases, the top and/or bottom substrate of a droplet actuator includes a glass or silicon substrate on which features have been patterned using process technology borrowed from semiconductor device fabrication including the deposition and etching of thin layers of materials using microlithography. The top and/or bottom substrate may consist of a semiconductor backplane (i.e., a thin-film transistor (TFT) active-matrix controller) on which droplet operations electrodes have been formed.

Electrodes of a droplet actuator are typically controlled by a controller or a processor, which is itself provided as part of a system, which may include processing functions as well as data and software storage and input and output capabilities. Reagents may be provided on the droplet actuator in the droplet operations gap or in a reservoir fluidly coupled to the droplet operations gap. The reagents may be in liquid form, e.g., droplets, or they may be provided in a reconstitutable form in the droplet operations gap or in a reservoir fluidly coupled to the droplet operations gap. Reconstitutable reagents may typically be combined with liquids for reconstitution.

The term “droplet operation” means any manipulation of a droplet on a droplet actuator. A droplet operation may, for example, include: loading a droplet into the droplet actuator; dispensing one or more droplets from a source droplet; splitting, separating or dividing a droplet into two or more droplets; transporting a droplet from one location to another in any direction; merging or combining two or more droplets into a single droplet; diluting a droplet; mixing a droplet; agitating a droplet; deforming a droplet; retaining a droplet in position; incubating a droplet; heating a droplet; vaporizing a droplet; cooling a droplet; disposing of a droplet; transporting a droplet out of a droplet actuator; other droplet operations described herein; and/or any combination of the foregoing. The terms “merge,” “merging,” “combine,” “combining” and the like are used to describe the creation of one droplet from two or more droplets. It should be understood that when such a term is used in reference to two or more droplets, any combination of droplet operations that are sufficient to result in the combination of the two or more droplets into one droplet may be used. For example, “merging droplet A with droplet B,” can be achieved by transporting droplet A into contact with a stationary droplet B, transporting droplet B into contact with a stationary droplet A, or transporting droplets A and B into contact with each other. The terms “splitting,” “separating” and “dividing” are not intended to imply any particular outcome with respect to volume of the resulting droplets (i.e., the volume of the resulting droplets can be the same or different) or number of resulting droplets (the number of resulting droplets may be 2, 3, 4, 5 or more). The term “mixing” refers to droplet operations which result in more homogenous distribution of one or more components within a droplet. Examples of “loading” droplet operations include microdialysis loading, pressure assisted loading, robotic loading, passive loading, and pipette loading. Droplet operations may be electrode-mediated. In some cases, droplet operations are further facilitated by the use of hydrophilic and/or hydrophobic regions on surfaces and/or by physical obstacles. Impedance and/or capacitance sensing and/or imaging techniques may sometimes be used to determine or confirm the outcome of a droplet operation. Generally speaking, the sensing or imaging techniques may be used to confirm the presence or absence of a droplet at a specific electrode. For example, the presence of a dispensed droplet at the destination electrode following a droplet dispensing operation confirms that the droplet dispensing operation was effective. Similarly, the presence of a droplet at a detection spot at an appropriate step in an assay protocol may confirm that a previous set of droplet operations has successfully produced a droplet for detection. Droplet transport time can be quite fast. For example, in various embodiments, transport of a droplet from one electrode to the next may be completed within about 1 sec, or about 0.1 sec, or about 0.01 sec, or about 0.001 sec. In one embodiment, the electrode is operated in AC mode but is switched to DC mode for imaging. It is helpful for conducting droplet operations for the footprint area of droplet to be similar to or larger than the electrowetting area; in other words, 1×-, 2×-3×-droplets are usefully controlled and/or operated using 1, 2, and 3 electrodes, respectively. If the droplet footprint is greater than number of electrodes available for conducting a droplet operation at a given time, the difference between the droplet size and the number of electrodes should typically not be greater than 1; in other words, a 2× droplet is usefully controlled using 1 electrode and a 3× droplet is usefully controlled using 2 electrodes. When droplets include beads, it is useful for droplet size to be equal to the number of electrodes controlling the droplet, e.g., transporting the droplet.

The term “filler fluid” means a fluid associated with a droplet operations substrate of a droplet actuator, which fluid is sufficiently immiscible with a droplet phase to render the droplet phase subject to electrode-mediated droplet operations. For example, the droplet operations gap of a droplet actuator is typically filled with a filler fluid. The filler fluid may, for example, be or include a low-viscosity, low-surface tension oil, such as silicone oil or hexadecane. The filler fluid may be or include a halogenated oil, such as a fluorinated or perfluorinated oil. The filler fluid may fill the entire gap of the droplet actuator or may only coat one or more surfaces of the droplet actuator or may only surround a droplet (i.e., an “oil-shell”) and the droplet brings its own oil with it. Filler fluids may be selected to improve droplet operations and/or reduce loss of reagent or target substances from droplets, reduce formation of unwanted microdroplets, reduce cross contamination between droplets, reduce contamination of droplet actuator surfaces, reduce degradation of droplet actuator materials, reduce evaporation of droplets, and so on. For example, filler fluids may be selected for compatibility with droplet actuator materials. As an example, fluorinated filler fluids may be usefully employed with fluorinated surface coatings. In another example, fluorinated filler fluids may be used to dissolve surface coatings (e.g., Fluorinert fc-40 may be a solvent for Teflon AF). Fluorinated filler fluids are useful to reduce loss of lipophilic compounds, such as umbelliferone substrates like 6-hexadecanoylamido-4-methylumbelliferone substrates (e.g., for use in Krabbe, Niemann-Pick, or other assays); Filler fluids may, for example, be doped with surfactants or other additives. For example, additives may be selected to improve droplet operations and/or reduce loss of reagent or target substances from droplets, formation of microdroplets, cross contamination between droplets, contamination of droplet actuator surfaces, degradation of droplet actuator materials, etc. Composition of the filler fluid, including surfactant doping, may be selected for performance with reagents or samples used in the specific assay protocols and effective interaction or non-interaction with droplet actuator materials. For example, fluorinated oils may in some cases be doped with fluorinated surfactants, e.g., Zonyl FSO-100 (Sigma-Aldrich) and/or others.

The term “reservoir” means an enclosure or partial enclosure configured for holding, storing, or supplying liquid. A droplet actuator system of the disclosure may include on-cartridge reservoirs and/or off-cartridge reservoirs. On-cartridge reservoirs may be (1) on-actuator reservoirs, which are reservoirs in the droplet operations gap or on the droplet operations surface; (2) off-actuator reservoirs, which are reservoirs on the droplet actuator cartridge, but outside the droplet operations gap, and not in contact with the droplet operations surface; or (3) hybrid reservoirs which have on-actuator regions and off-actuator regions. An example of an off-actuator reservoir is a reservoir in the top substrate. An off-actuator reservoir is typically in fluid communication with an opening or flow path arranged for flowing liquid from the off-actuator reservoir into the droplet operations gap, such as into an on-actuator reservoir. An off-cartridge reservoir may be a reservoir that is not part of the droplet actuator cartridge at all, but which flows liquid to some portion of the droplet actuator cartridge. For example, an off-cartridge reservoir may be part of a system or docking station to which the droplet actuator cartridge is coupled during operation. Similarly, an off-cartridge reservoir may be a reagent storage container or syringe which is used to force fluid into an on-cartridge reservoir or into a droplet operations gap. A system using an off-cartridge reservoir will typically include a fluid passage means whereby liquid may be transferred from the off-cartridge reservoir into an on-cartridge reservoir or into a droplet operations gap.

The term “washing” with respect to washing a surface, such as a hydrophilic surface, means reducing the amount and/or concentration of one or more substances in contact with the surface or exposed to the surface from a droplet in contact with the surface. The reduction in the amount and/or concentration of the substance may be partial, substantially complete, or even complete. The substance may be any of a wide variety of substances; examples include target substances for further analysis, and unwanted substances, such as components of a sample, contaminants, and/or excess reagent or buffer.

The terms “top,” “bottom,” “over,” “under,” and “on” are used throughout the description with reference to the relative positions of components of the droplet actuator, such as relative positions of top and bottom substrates of the droplet actuator. It will be appreciated that in many cases the droplet actuator is functional regardless of its orientation in space.

When a liquid in any form (e.g., a droplet or a continuous body, whether moving or stationary) is described as being “on”, “at”, or “over” an electrode, array, matrix or surface, such liquid could be either in direct contact with the electrode/array/matrix/surface, or could be in contact with one or more layers or films that are interposed between the liquid and the electrode/array/matrix/surface. In one example, filler fluid can be considered as a dynamic film between such liquid and the electrode/array/matrix/surface.

When a droplet is described as being “on” or “loaded on” a droplet actuator, it should be understood that the droplet is arranged on the droplet actuator in a manner which facilitates using the droplet actuator to conduct one or more droplet operations on the droplet, the droplet is arranged on the droplet actuator in a manner which facilitates sensing of a property of or a signal from the droplet, and/or the droplet has been subjected to a droplet operation on the droplet actuator.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The terms “comprise,” “comprises,” “comprising,” “have,” “has,” “having” “include,” “includes,” and “including,” are intended to be non-limiting (i.e., open ended), such that recitation of items in a list is not to the exclusion of other like items that may be substituted or added to the listed items (i.e., not closed).

Terms like “preferably,” “commonly,” and “typically” are not utilized herein to limit the scope of the claimed embodiments or to imply that certain features are critical or essential to the structure or function of the claimed embodiments. These terms are intended to highlight alternative or additional features that may or may not be utilized in a particular embodiment of the disclosure.

The term “substantially” is utilized herein to represent the inherent degree of uncertainty that may be attributed to any quantitative comparison, value, measurement, or other representation and to represent the degree by which a quantitative representation may vary from a stated reference without resulting in a change in the basic function of the subject matter at issue.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### Linear or One-Dimensional (1D) Ganged Reservoir Configurations

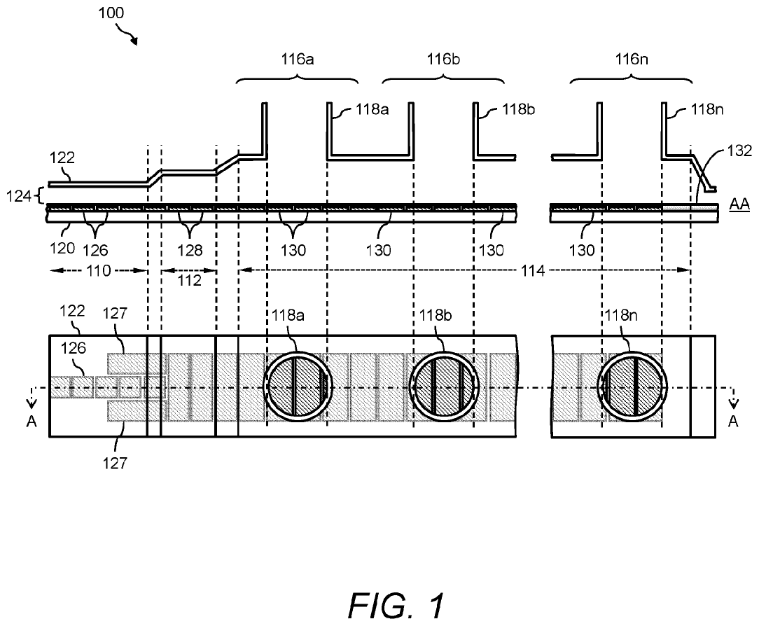

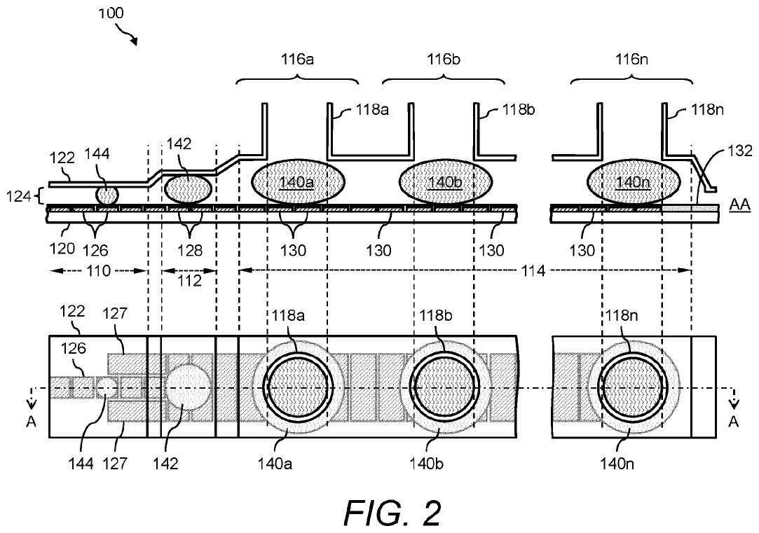

FIG. 1 and FIG. 2 are plan views and cross-sectional views of an example of a linear ganged (or extended) reservoir configuration 100 including multiple storage locations and multiple loading ports, in accordance with an embodiment of the disclosure. Further, as described hereinbelow, a ganged reservoir (or well) may be defined to include a dispenser portion, a primer portion, and a reservoir portion. For example, as described hereinbelow, the ganged reservoir (or well) may include a dispenser portion 110 may be supplied by a primer portion 112 and primer portion 112 may be supplied by a reservoir portion 114. Further, reservoir portion 114 may include any arrangement of two or more storage locations 116.

The physical structure of linear ganged reservoir configuration 100 may be generally that of a microfluidics device, which may be, for example, a digital microfluidics (DMF) device. For example, linear ganged reservoir configuration 100 may include a bottom substrate 120 and a top substrate 122 separated by a droplet operations gap 124. In the case of DMF, droplet operations may occur in the droplet operations gap 124 between bottom substrate 120 and top substrate 122 of linear ganged reservoir configuration 100.

In one example, bottom substrate 120 may be a printed circuit board (PCB)-based substrate, such as a multi-layer PCB. In another example, bottom substrate 120 may be glass or silicon substrate that has patterned electrodes. In one example, top substrate 122 may be formed of glass or plastic. For example, top substrate 122 may be formed of injection molded thermoplastic materials or injection molded glass. Additionally, top substrate 122 may be substantially transparent to light. However, in other embodiments, top substrate 122 may be substantially opaque. Additionally, top substrate 122 may include a ground or reference electrode (not shown). In one example, the ground or reference electrode may be formed of indium tin oxide (ITO). In some embodiments, the ITO ground or reference electrode is substantially transparent to light.

In linear ganged reservoir configuration 100, dispenser portion 110 may include multiple lines, paths, and/or arrays of droplet operations electrodes 126 (e.g., electrowetting electrodes). Additionally, certain droplet operations electrodes 126 may be flanked by electrodes 127. Further, primer portion 112 may include multiple lines, paths, and/or arrays of primer electrodes 128 (e.g., electrowetting electrodes). Further, reservoir portion 114 may include multiple lines, paths, and/or arrays of reservoir electrodes 130 (e.g., electrowetting electrodes). Additionally, reservoir portion 114 may include at least two storage locations 116 up to any number of storage locations 116 (e.g., storage locations 116a through 116n) all ganged together. In this example, storage location 116a may be considered the outlet storage location 116 (i.e., storage location proximal to the primer portion 112). Further, in this example, each of the storage locations 116 has a loading port 118 (e.g., loading ports 118a through 118n). The loading port 118 allows a user of the device to provide a fluid to the storage locations 116, such as, for example, by pipetting. The loading port 118 may have a similar size and or geometry to the wells of a standard microwell plate. In configurations where each storage location 116 has a loading port 118, each storage location 116 may be filled without activating the electrodes to move droplets between storage locations 116. Droplet operations electrodes 126, primer electrodes 128, and reservoir electrodes 130 may be formed, for example, of copper, gold, chromium, or aluminum.

A dielectric layer 132 (e.g., parylene coating, silicon nitride) may be atop the electrodes 126, 128, and 130. Further, a hydrophobic layer (not shown) may be provided on both the top side of bottom substrate 120 and the bottom side of top substrate 122 that is facing droplet operations gap 124. Examples of hydrophobic materials or coatings may include, but are not limited to, polytetrafluoroethylene (PTFE), Cytop, Teflon™ AF (amorphous fluoropolymer) resins, FluoroPel™ coatings, silane, and the like.

In linear ganged reservoir configuration 100, to accommodate different volumes of liquid, the height of droplet operations gap 124 may vary from dispenser portion 110, to primer portion 112, to reservoir portion 114. For example, each storage location 116 of reservoir portion 114 may be designed to hold a discrete large-volume droplet 140 (see FIG. 2). In some embodiments, the large-volume droplet 140 may have a volume from about 20 μL to about 500 μL. In other embodiments, the large-volume droplet 140 may have a volume from about 100 μL to about 5000 μL. Further, primer portion 112 may be designed to hold a mid-volume droplet 142 (see FIG. 2). In some embodiments, the mid-volume droplet 142 may have a volume from about 5 μL to about 100 μL. In other embodiments, the mid-volume droplet 142 may have a volume from about 20 μL to about 200 μL. Further, dispenser portion 110 may be designed to hold a small-volume droplet 144 (see FIG. 2). In some embodiments, the small-volume droplet 144 may have a volume from about 1 μL to about 10 μL. In other embodiments, the small-volume droplet 144 may have a volume from about 2 μL to about 4 μL.

Accordingly, at reservoir portion 114, the gap height of droplet operations gap 124 may be, for example, from about 300 μm to about 3000 μm. At primer portion 112, the gap height of droplet operations gap 124 may be, for example, from about 100 μm to about 1000 μm. At dispenser portion 110, the gap height of droplet operations gap 124 may be, for example, from about 50 μm to about 500 μm.

Further, in linear ganged reservoir configuration 100, primer portion 112 and dispenser portion 110 may be designed or sized optimally for very precise dispensing. For example, each of the droplet operations electrodes 126 may have the following dimensions: 1.25 mm×1.25 mm; 1.25 mm×3 mm; 1 mm×1 mm; 1.25 mm×3.3 mm; or 1.25 mm×3.85 mm.

Each of the storage locations 116 of reservoir portion 114 may be designed to hold, for example, from about 800 μL to about 1000 μL, or from about 1000 μL to about 1500 μL, or from about 2.5 mL to about 5 mL. When, for example, a certain larger volume of liquid is needed, then a line of multiple storage locations 116 may be provided. In one example, linear ganged reservoir configuration 100 may include a line of 1500 μL storage locations 116 to provide some greater volume of liquid. In another example, linear ganged reservoir configuration 100 may include a line of 2-5 μL storage locations 116 to provide some greater volume of liquid.

FIG. 1 shows linear ganged reservoir configuration 100 absent liquid. By contrast, FIG. 2 shows linear ganged reservoir configuration 100 loaded with liquid. For example, each of the storage locations 116 of reservoir portion 114 may hold a discrete large-volume droplet 140 (e.g., large-volume droplets 140a through 140n). Each large-volume droplet 140 is maintained separately in its respective storage location 116 and does not touch any neighboring large-volume droplet 140 in a neighboring storage location 116.

In operation, each of the storage locations 116a through 116n of reservoir portion 114 may be loaded with a large-volume droplet 140 via the respective loading ports 118a through 118n. The primer portion 112 may prime the large-volume droplet 140 using droplet operations, such that mid-volume droplets 142 may be split off from a large-volume droplet 140 at storage location 116a and dispensed into primer portion 112. Priming may also include manipulating a fluid within the reservoir portion 114 to ensure it is the correct shape and that the exterior of the fluid is equilibrated to a surfactant in the oil phase.

Then, small-volume droplets 144 may be split off from a mid-volume droplet 142 and dispensed into dispenser portion 110. Then, using droplet operations, small-volume droplets 144 may be transported away for processing. As, for example, the large-volume droplet 140 at storage location 116a is depleted, the next large-volume droplet 140 may be transported (via droplet operations) into storage location 116a from storage location 116b. Then, all other upstream large-volume droplets 140 may also be advanced (via droplet operations) to the next storage location 116 toward primer portion 112. This process may continue until all large-volume droplets 140a through 140n are substantially depleted or consumed.

Further, after substantially depleting and/or consuming large-volume droplets 140a through 140n, there may be some small residue left within any storage location 116. In one example, this residue may be simply left and held within storage location 116. In another example, assuming a large enough volume, this residue may be transported (via droplet operations) out of storage location 116 to waste.

That is, using linear ganged reservoir configuration 100, multiple large-volume droplets 140 at multiple storage locations 116 may be used to resupply mid-volume droplets 142 at the primer portion 112 and then resupply small-volume droplets 144 at the dispenser portion 110. Then, small-volume droplets 144 from dispenser portion 110 may be used to supply any processes of a microfluidics device (not shown).

In microfluidics certain surface forces (e.g., surface tension) and certain volume forces (e.g., gravity) should be balanced to enable electrowetting. Therefore, it is important to note that the maximum volume of any large-volume droplet 140 must be just short of any volume that causes gravity to flood the system. At the same time, if the volume of large-volume droplet 140 is too small, then it may be difficult to split off a mid-volume droplet 142 into primer portion 112 (using droplet operations). Accordingly, large-volume droplet 140 at each storage location 116 may have a certain optimal maximum-minimum volume which may depend in part on the size of the reservoir portion 114 and the size and shape of the reservoir electrodes 148.

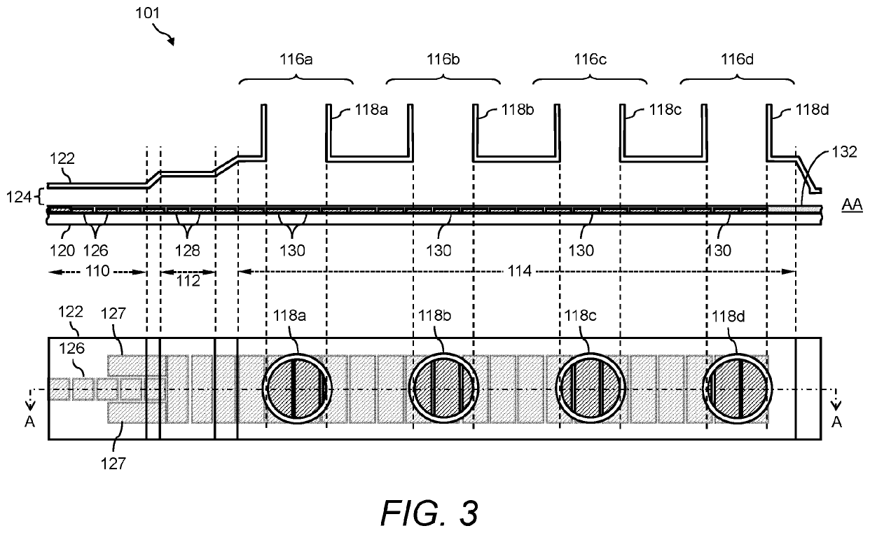

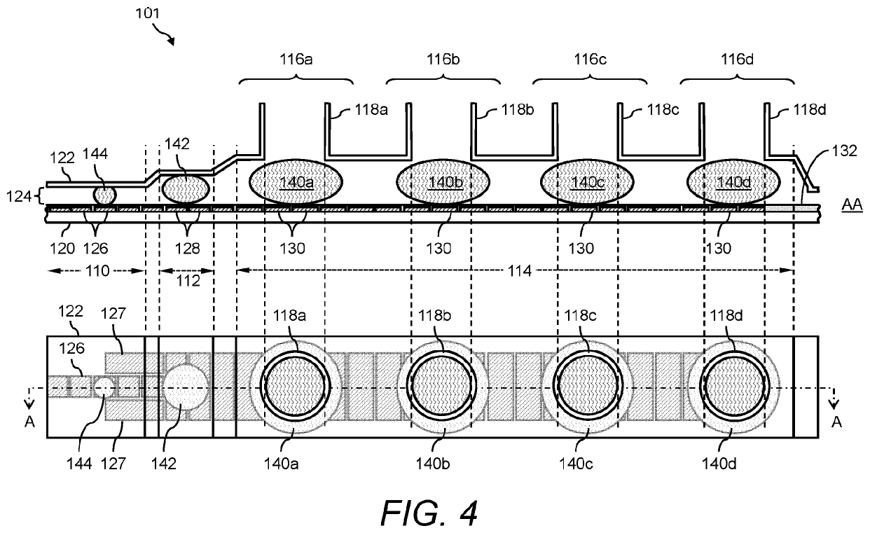

FIG. 3 and FIG. 4 are plan views and cross-sectional views of an example of a linear ganged reservoir configuration 100 including four storage locations 116 and four loading ports 118, in accordance with an embodiment of the disclosure. For example, ganged reservoir configuration 100 may provide an m×n arrangement of storage locations 116 and loading ports 118. FIG. 3 and FIG. 4 show a specific example of the linear ganged reservoir configuration 100 shown in FIG. 1 and FIG. 2, which can be called a 4×4-linear ganged reservoir 101, in that it comprises 4 storage locations and 4 loading ports. That is, in this example, the 4×4-linear ganged reservoir 101 may include storage locations 116a, 116b, 116c, and 116d and respective loading ports 118a, 118b, 118c, and 118d. In 4×4-linear ganged reservoir 101, storage location 116a may be considered the outlet storage location 116 (i.e., storage location proximal to the primer portion 112). FIG. 3 shows 4×4-linear ganged reservoir 101 absent liquid, while FIG. 4 shows 4×4-linear ganged reservoir 101 loaded with liquid.

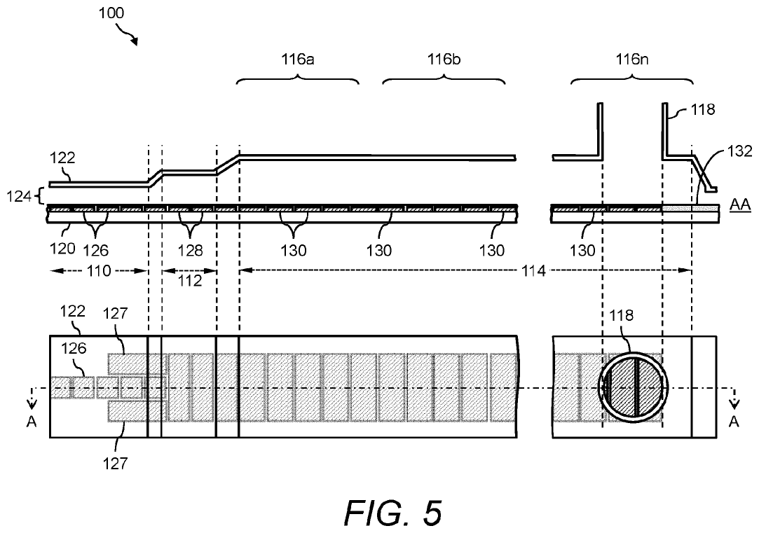

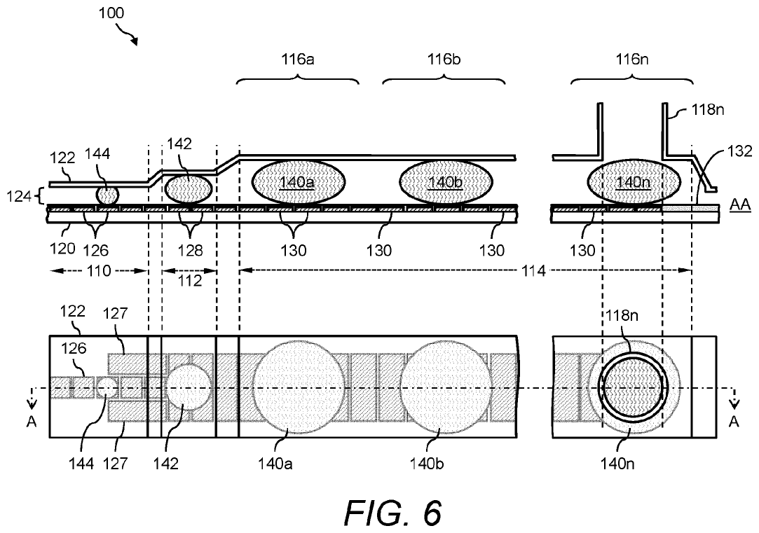

FIG. 5 and FIG. 6 are plan views and cross-sectional views of an example of a linear ganged reservoir configuration 100 including multiple storage locations 116 and one loading port 118, in accordance with an embodiment of the disclosure. That is, linear ganged reservoir configuration 100 shown in FIG. 5 and FIG. 6 may be substantially the same as the linear ganged reservoir configuration 100 shown in FIG. 1 and FIG. 2 except that it has one loading port 118 only instead of each storage location 116 having its own loading port 118. In this example, the one loading port 118 is located at the storage location 116n distal from primer portion 112 and dispenser portion 110. Again, storage location 116a may be considered the outlet storage location 116 (i.e., storage location proximal to the primer portion 112).

Further, the operation of linear ganged reservoir configuration 100 shown in FIG. 5 and FIG. 6 may be substantially the same as the linear ganged reservoir configuration 100 shown in FIG. 1 and FIG. 2 except that all storage locations 116 are filled using the one loading port 118 located at the storage location 116n (distal from primer portion 112). That is, storage location 116n is filled, then the large-volume droplet 140 is transported via droplet operations to a different storage location 116. This process may be repeated until all storage locations 116 are filled. Unlike the example provided in FIG. 3 and FIG. 4, the example provided in FIG. 5 and FIG. 6 requires activation of the electrodes to move droplets between storage locations 116.

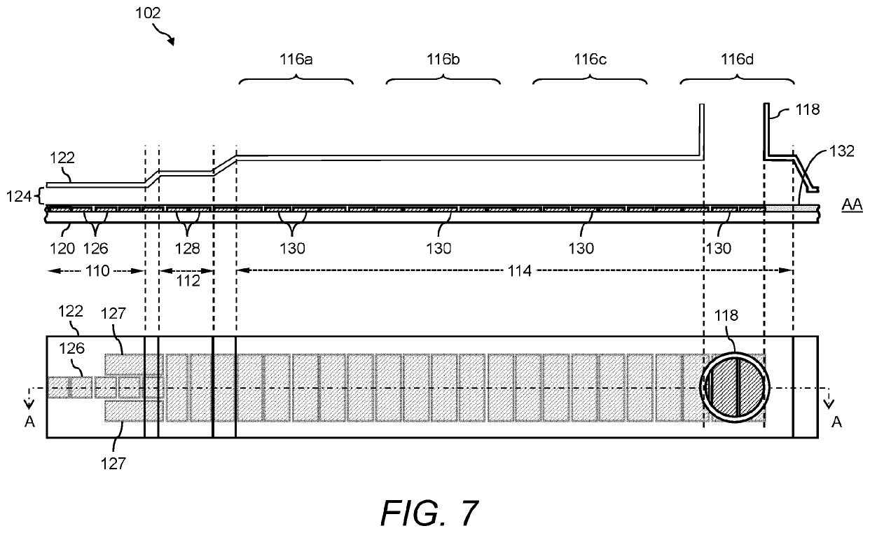

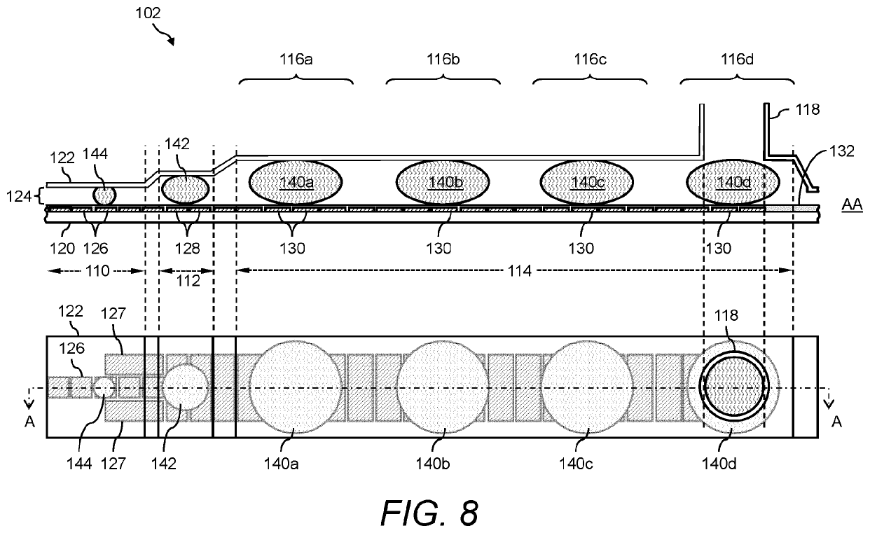

FIG. 7 and FIG. 8 are plan views and cross-sectional views of an example of a linear ganged reservoir configuration 100 including four storage locations 116 and one loading port 118, in accordance with an embodiment of the disclosure. FIG. 7 and FIG. 8 show a specific example of the linear ganged reservoir configuration 100 shown in FIG. 5 and FIG. 6, which can be called a 4×1-linear ganged reservoir 102, in that it comprises 4 storage locations and 1 loading port. That is, in this example, the 4×1-linear ganged reservoir 102 may include storage locations 116a, 116b, 116c, and 116d and one loading port 118 at storage location 116d. In 4×1-linear ganged reservoir 102, storage location 116a may be considered the outlet storage location 116 (i.e., storage location proximal to the primer portion 112). FIG. 7 shows 4×1-linear ganged reservoir 101 absent liquid, while FIG. 8 shows 4×1-linear ganged reservoir 102 loaded with liquid.

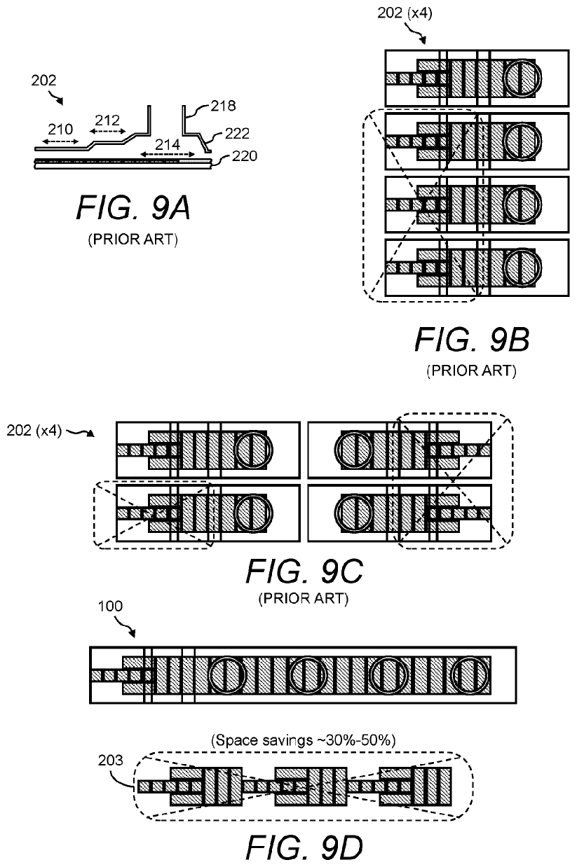

By way of contrast, FIG. 9A shows a cross-sectional view of an example of a conventional well or reservoir 202 for holding liquids. In this example, the conventional well or reservoir 202 may include one storage location only and one loading port only. For example, conventional well or reservoir 202 may include a dispenser portion 210, a primer portion 212, and a reservoir portion 214 that includes one storage location with its loading port 218. Conventional well or reservoir 202 may include a bottom substrate 220 and a top substrate 222.

FIG. 9B and FIG. 9C are plan views of example configurations of conventional wells or reservoirs. For example, FIG. 9B shows an example of four conventional wells or reservoirs 202 arranged in parallel. Further, FIG. 9C shows an example of four conventional wells or reservoirs 202 arranged oppositely of one another. Further, FIG. 9D shows that by replacing the four conventional wells or reservoirs 202 with, for example, the 4×4-linear ganged reservoir 101 shown in FIG. 3 and FIG. 4 that three of the four primer/dispenser portions of the four conventional wells or reservoirs 202 may be eliminated.

In this example, the use of 4×4-linear ganged reservoir 101 in place of the four conventional wells or reservoirs 202 may result in a space savings of, for example, from about 30% to about 50%. At the same time, 4×4-linear ganged reservoir 101 may store substantially the same volume of liquid as the four conventional wells or reservoirs 202, while requiring less real-estate on the microfluidics device. That is, 4×4-linear ganged reservoir 101 provides substantially the same amount of liquid storage as the four conventional wells or reservoirs 202 while having a smaller footprint than the four conventional wells or reservoirs 202.

### Two-Dimensional (2D) Ganged Reservoir Configurations

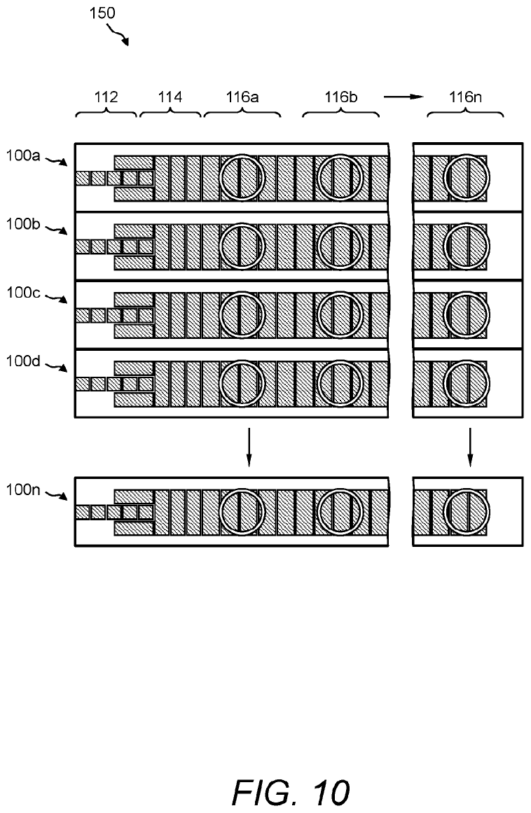

FIG. 10 is a plan view of an example of a ganged reservoir configuration 150 including multiple linear ganged reservoir configurations 100 arranged in parallel, in accordance with an embodiment of the disclosure. For example, ganged reservoir configuration 150 may provide an l×m×n arrangement of storage locations 116 and loading ports 118. That is, ganged reservoir configuration 150 may include l-number of linear ganged reservoir configurations 100. Each linear ganged reservoir configuration 100 may include m-number of storage locations 116. Each linear ganged reservoir configuration 100 may include n-number of loading ports 118. In each linear ganged reservoir configuration 100, storage location 116a may be considered the outlet storage location 116 (i.e., storage location proximal to the primer portion 112).

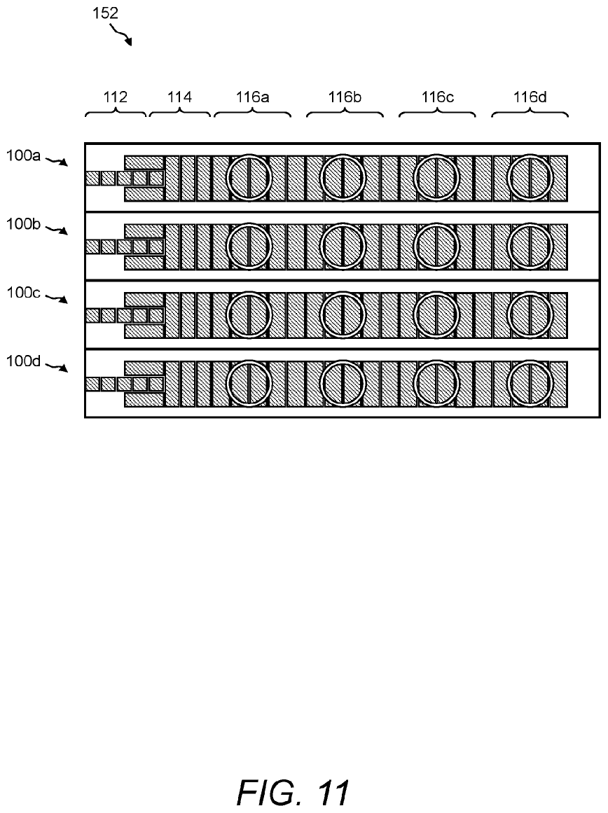

FIG. 11 is a plan view of an example of a 4×4×4-ganged reservoir 152 including four 4×4-linear ganged reservoirs 102 arranged in parallel, which is one example of the ganged reservoir configuration 150 shown in FIG. 10. For example, 4×4×4-ganged reservoir 152 may provide 16 storage locations 116 and 16 loading ports 118. That is, 4×4×4-ganged reservoir 152 may include four 4×4-linear ganged reservoirs 101 (see FIG. 3 and FIG. 4). Each 4×4-linear ganged reservoir 101 may include four storage locations 116 and four loading ports 118. Each 4×4-linear ganged reservoir 101, storage location 116a may be considered the outlet storage location 116 (i.e., storage location proximal to the primer portion 112).

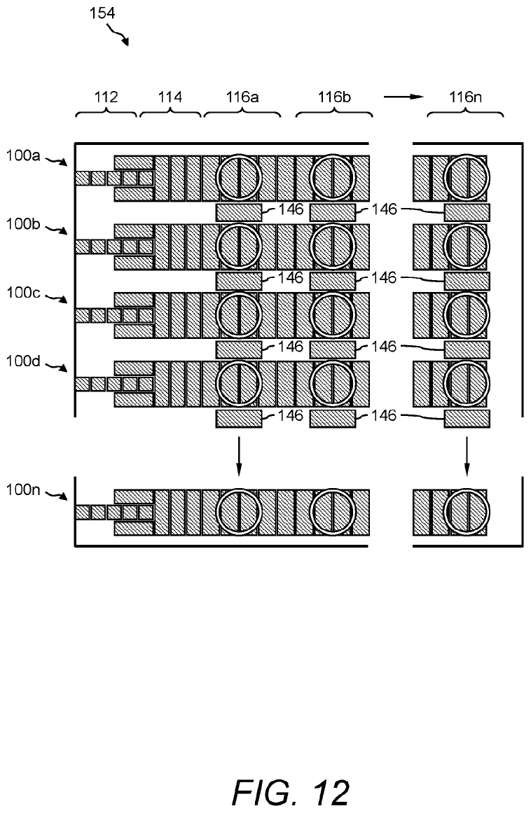

In the ganged reservoir configurations described hereinabove, the transport of large-volume droplets 140 from one storage location 116 to a different storage location 116 is limited to linear transport. However, the ganged reservoir configurations are not limited to linear movement only of large-volume droplets 140. In other embodiments, large-volume droplets 140 may be transported linearly and also across lanes of storage locations 116. That is, when, for example, a certain larger volume of liquid is needed, then an array of multiple storage locations 116 may be provided. In one example, linear ganged reservoir configuration 100 may include an array of 1500 μL storage locations 116 to provide some greater volume of liquid. In another example, linear ganged reservoir configuration 100 may include an array of 2-5 μL storage locations 116 to provide some greater volume of liquid. Examples of which are described hereinbelow with reference to FIG. 12 through FIG. 19.

FIG. 12 is a plan view of an example of a ganged-reservoir array configuration 154 including bridge electrodes for transporting liquids between lanes of linear ganged reservoir configurations 100 and including multiple outlet storage locations 116, in accordance with an embodiment of the disclosure. For example, ganged-reservoir array configuration 154 may be substantially the same as ganged reservoir configuration 150 shown in FIG. 10 except for the addition of bridge electrodes 146 arranged between lanes of linear ganged reservoir configurations 100. In this example, bridge electrodes 146 may be substantially rectangular-shaped. In this example, the bridge electrodes 146 may be positioned between adjacent storage locations 116. In ganged-reservoir array configuration 154, each storage location 116a of each linear ganged reservoir configuration 100 may be considered an outlet storage location 116 (i.e., storage location proximal to the primer portion 112). Accordingly, ganged-reservoir array configuration 154 may include multiple outlet storage locations 116.

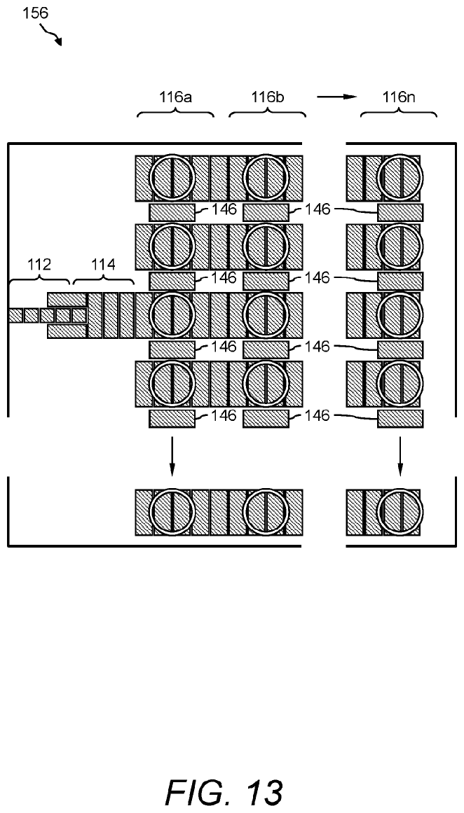

FIG. 13 is a plan view of an example of a ganged-reservoir array configuration 156 including bridge electrodes 146 for transporting liquids between lanes of storage locations 116 and including one outlet storage location 116 (i.e., storage location proximal to the primer portion 112), in accordance with an embodiment of the disclosure. For example, ganged-reservoir array configuration 156 may be substantially the same as ganged-reservoir array configuration 154 shown in FIG. 12 except for the absence of multiple outlet storage locations 116. That is, ganged-reservoir array configuration 156 may include an l×m×n array of storage locations 116. Further, in this example, only one lane of storage locations 116 includes primer portion 112 and dispenser portion 110. However, each of the storage locations 116 may supply the lane comprising the primer portion 112 and dispenser portion 110.

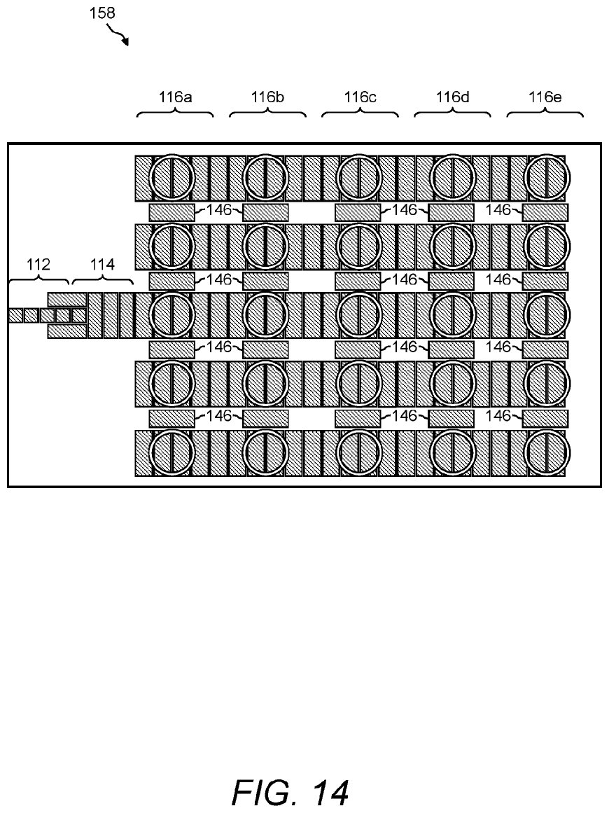

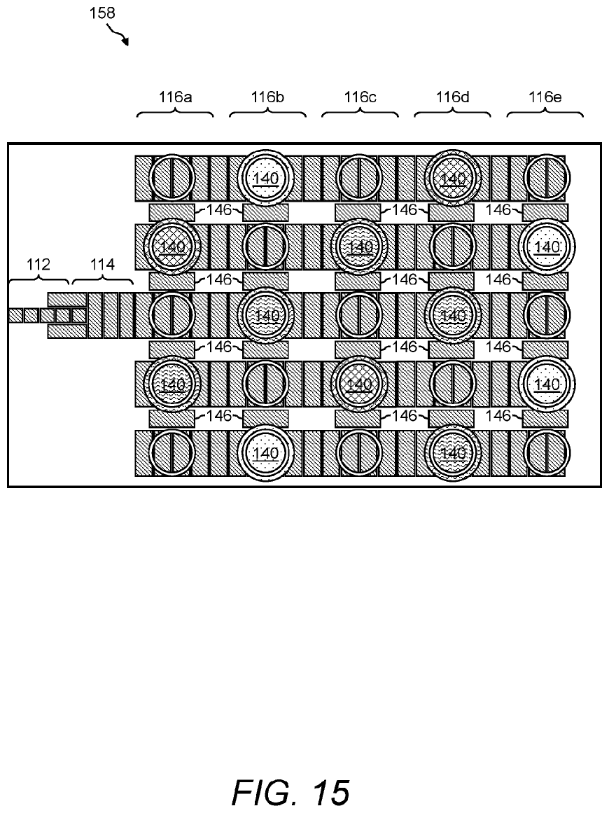

FIG. 14 and FIG. 15 are plan views of an example of a 5×5×5 ganged-reservoir array 158 including one outlet storage location 116 (i.e., storage location proximal to the primer portion 112), in accordance with an embodiment of the disclosure. The 5×5×5 ganged-reservoir array 158 is one example of the ganged-reservoir array configuration 156 shown in FIG. 13. In this example, 5×5×5 ganged-reservoir array 158 may include 25 storage locations 116 and 25 loading ports 118. Further, each of the outer storage locations 116 may supply the centermost line of storage locations 116 which in turn may supply primer portion 112 and dispenser portion 110. The storage location 116 supplying primer portion 112 and dispenser portion 110 may be considered the outlet storage location 116 (i.e., storage location proximal to the primer portion 112).

FIG. 14 shows a 5×5×5 ganged-reservoir array 158 absent liquid, while FIG. 15 shows a 5×5×5 ganged-reservoir array 158 loaded with liquid. For example, FIG. 15 shows that some storage locations 116 may be holding a large-volume droplet 140 while other storage locations 116 may be empty. Additionally, the 5×5×5 ganged-reservoir array 158 may be loaded with different types of large-volume droplets 140. For example, some large-volume droplets 140 may be buffer droplets, others reagent droplets, others wash droplets, and so on. Accordingly, the volume, composition, and/or order of the large-volume droplets within the reservoir portion 114 may be varied and may be dependent on the processes being performed on the DMF device.

Using droplet operations, large-volume droplets 140 may be moved from one storage location 116 to a different storage location 116. Empty storage locations 116 may be provided to allow movement or shuffling of large-volume droplets 140 to or from different storage locations 116. For example, to minimize contamination between different types of large-volume droplets 140, a large-volume wash droplet 140 may be transported across any shared reservoir electrodes 130 for cleaning purposes. Although, different types of large-volume droplets 140 may be provided, the different types of large-volume droplets 140 should be provided in an order that supports the processes of the microfluidics device and minimizes cross-contamination.

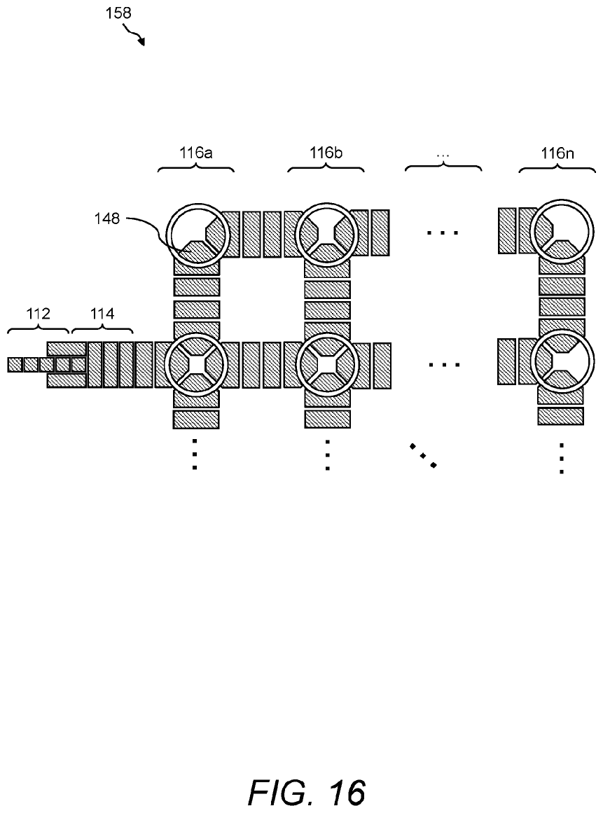

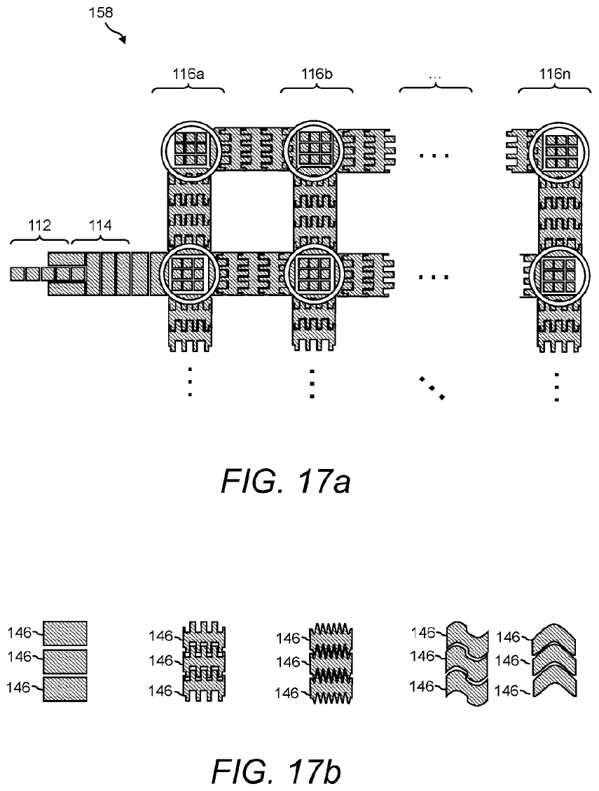

FIG. 16 and FIG. 17a are plan views of examples of ganged-reservoir array configurations including multiple bridge electrodes and/or different shaped bridge electrodes, and/or different shape reservoir electrodes, in accordance with an embodiment of the disclosure. In one example, FIG. 16 shows a ganged-reservoir array 158 including two substantially rectangular bridge electrodes 146 arranged between each of the storage locations 116. Each of the storage locations 116 feature from two to four reservoir electrodes 148 having an isosceles trapezoid shape. The reservoir electrodes 148 are connected to bridge electrodes 146 at their base.

In another example, FIG. 17a shows a ganged-reservoir array 158 including bridge electrodes 146 having unique shapes. Ganged-reservoir array 158 may include any number and/or any shapes of bridge electrodes 146 arranged between lanes of storage locations 116 (as shown in FIG. 17b). For example, bridge electrodes 146 may be rectangular, interdigitated square waves or sinus waves, free form or concave/convex. The bridge electrodes 146 may provide either unidirectional or bidirectional transport of a droplet. For example, concave bridge electrodes 146 may provide unidirectional transport of a droplet.

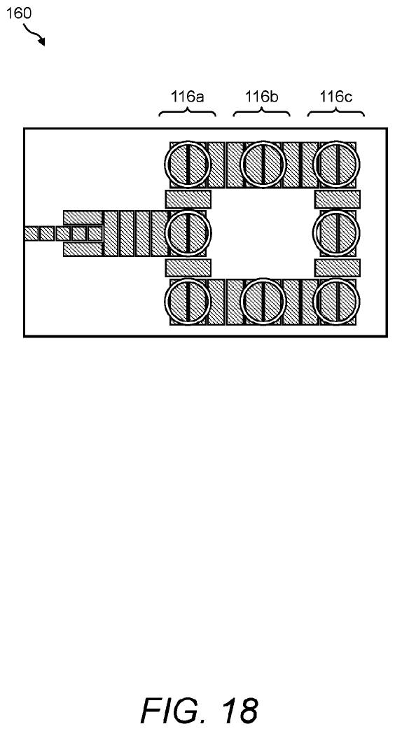

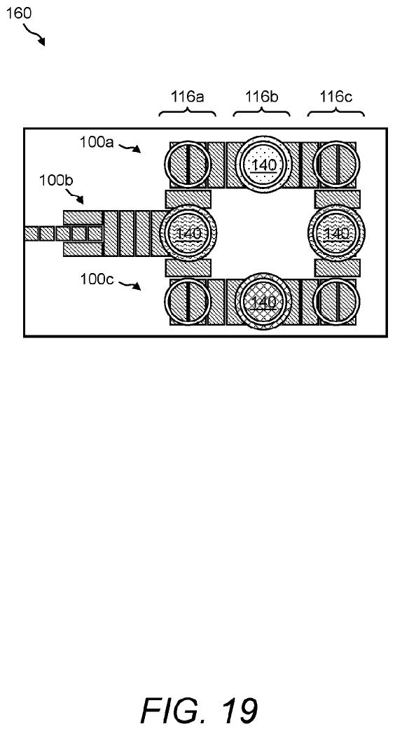

FIG. 18 and FIG. 19 are plan views of an example of a looped ganged reservoir 160, in accordance with an embodiment of the disclosure. In this example, looped ganged reservoir 160 may include, for example, eight storage locations 116 arranged in a loop as shown. However, this is exemplary only, and looped ganged reservoir 160 may include any number of storage locations 116 arranged in a loop. Further, one of storage locations 116 may supply primer portion 112 and dispenser portion 110. The storage location 116 supplying primer portion 112 and dispenser portion 110 may be considered the outlet storage location 116 (i.e., storage location proximal to the primer portion 112).

FIG. 18 shows looped ganged reservoir 160 absent liquid, while FIG. 19 shows looped ganged reservoir 160 loaded with liquid. For example, FIG. 19 shows that some storage locations 116 may be holding a large-volume droplet 140 while other storage locations 116 may be empty. Additionally, looped ganged reservoir 160 may be loaded with different types of large-volume droplets 140. For example, some large-volume droplets 140 may be buffer droplets, while other large-volume droplets 140 may be reagent droplets, wash droplets, or other types of droplets.

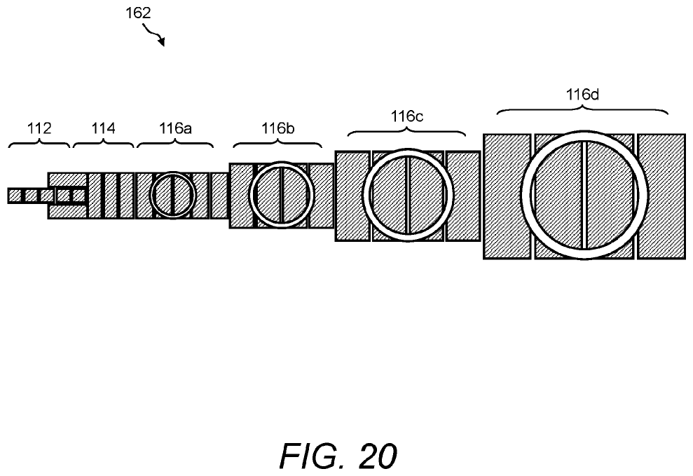

FIG. 20 is a plan view of an example of a cascading linear ganged reservoir 162 including different sized storage locations 116 arranged, for example, from small-volume to large-volume, in accordance with an embodiment of the disclosure. In one example, cascading linear ganged reservoir 162 may include four storage locations 116 (e.g., storage locations 116a, 116b, 116c, 116d). In this example, storage location 116d may be the largest volume storage location 116 while storage location 116a may be the smallest volume storage location 116. Further, storage location 116a supplies primer portion 112 and dispenser portion 110. Accordingly, storage location 116a may be the outlet storage location 116 (i.e., storage location proximal to the primer portion 112).

In one example, storage location 116d may hold a large-volume droplet 140 that may be from about 1000 μL to about 5000 μL in volume. Storage location 116c may hold a large-volume droplet 140 that may be from about 100 μL to about 500 μL in volume. Storage location 116b may hold a large-volume droplet 140 that may be from about 10 μL to about 50 μL in volume. Storage location 116a may hold a large-volume droplet 140 that may be from about 1 μL to about 5 μL in volume.

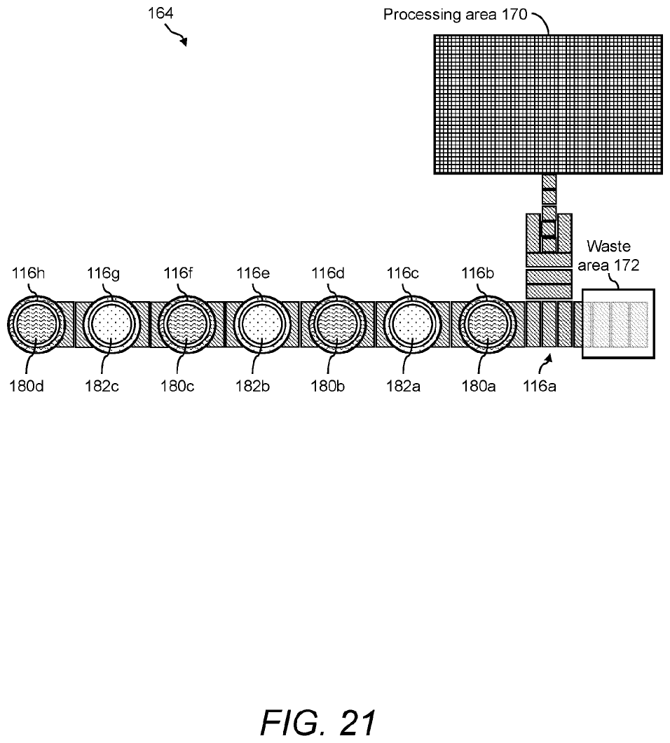

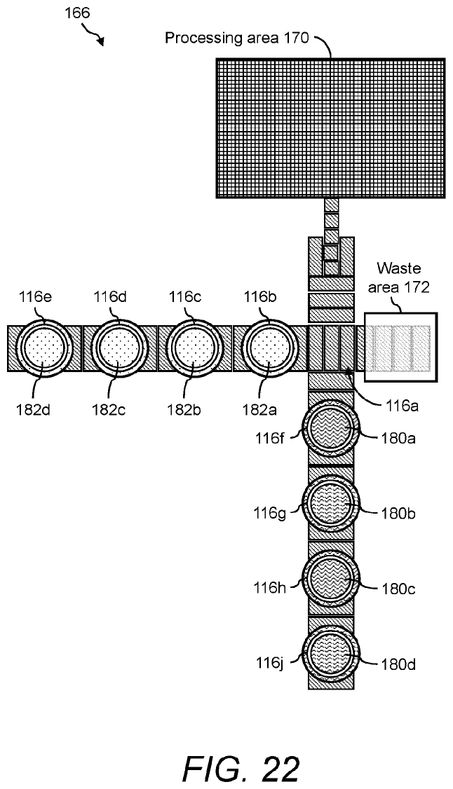

FIG. 21 and FIG. 22 are plan views of examples of electrode arrangements including linear ganged reservoirs in relation to a processing area and a waste area, in accordance with an embodiment of the disclosure. For example, FIG. 21 shows electrode configuration 164 and FIG. 22 shows alternative electrode configuration 166.

Electrode configuration 164 of FIG. 21 may include, for example, a storage location 116a that supplies primer portion 112 and dispenser portion 110 and wherein dispenser portion 110 further supplies a processing area 170. A line of other storage locations 116 supply one side of storage location 116a. For example, seven storage locations 116b through 116h supply storage location 116a. Accordingly, storage location 116a may be the outlet storage location 116. The arrangement of storage locations 116b through 116h along with storage location 116a supplying primer portion 112 and dispenser portion 110 is another example of a linear ganged reservoir configuration 100 shown in FIG. 1 and FIG. 2.

Processing area 170 may include any arrangements of any types of electrodes for performing any functions of a microfluidics device. For example, processing area 170 may include mixing regions, sensing regions, detection regions, and the like.

Further, the other side of storage location 116a supplies a waste area 172. In one example, every other storage location 116 may include one type of large-volume sample droplet 180 while the remaining storage locations 116 may include another type of large-volume droplet 182. For example, storage locations 116b, 116d, 116f, and 116h may include large-volume sample droplets 180a, 180b, 180c, and 180d, respectively. Further, storage locations 116c, 116e, and 116g may include other large-volume droplets 182a, 182b, and 182c, respectively.

During processing, any of one type of large-volume sample droplets 180 and/or another type of large-volume droplets 182 may be transported to storage location 116a and dispensed into processing area 170. However, at any given time, the entire volume at any droplet may not be used completely during dispensing. Therefore, there may be reason to move any unused volume to waste area 172. Accordingly, electrode configuration 164 facilitates both the advantages of linear ganged reservoir configuration 100 and the ability to move any unused volume to waste.

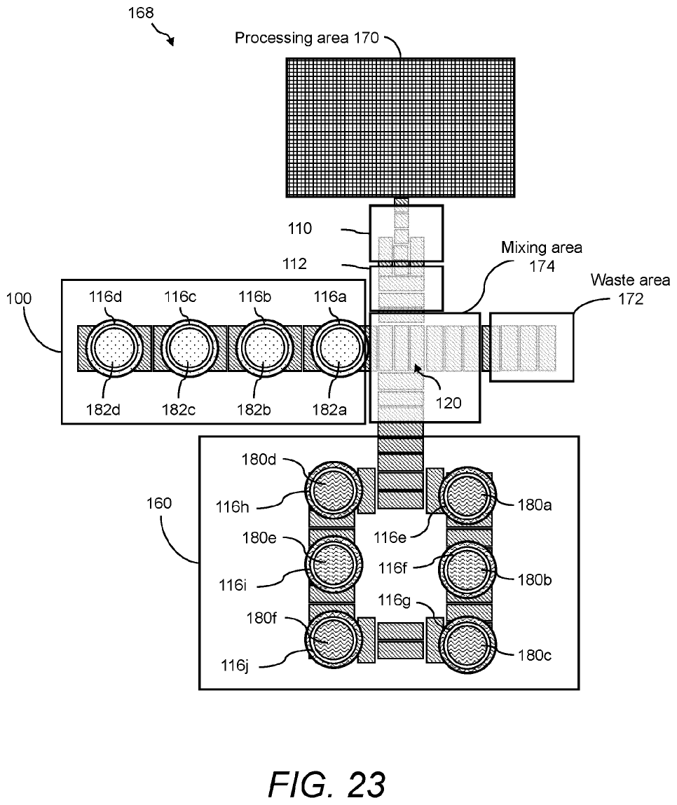

FIG. 23 is a plan view of examples of electrode arrangements including a linear ganged reservoir and looped ganged reservoir in relation to a mixing area, processing area and a waste area, in accordance with an embodiment of the disclosure. For example, FIG. 23 shows electrode configuration 168.

Electrode configuration 168 of FIG. 23 may include, for example, a mixing area 174 that supplies primer portion 112 and dispenser portion 110 and wherein dispenser portion 110 further supplies a processing area 170. A line of other storage locations 116 supply one side of mixing area 174. For example, four storage locations 116a through 116d supply mixing area 174. Accordingly, storage location 116a may be the outlet storage location 116 (i.e., storage location proximal to the primer portion 112) for the linear arrangement of storage locations 116. Additionally, electrode configuration 168 of FIG. 23 may include a looped arrangement of storage locations 116. The lopped arrangement of storage locations 116 may supply another side of mixing area 174. For example, six storage locations 116e through 116j supply mixing area 174. Accordingly, either storage location 116h or 116e may be the outlet storage location 116 (i.e., storage location proximal to the primer portion 112) for the looped arrangement of storage locations 116.

Processing area 170 may include any arrangements of any types of electrodes for performing any functions of a microfluidics device. For example, processing area 170 may include further mixing regions or sensing regions, detection regions, and the like.

Further, the other side of mixing area 174 supplies a waste area 172. In one example, the linear arrangement of storage locations 116 may include one type of large-volume sample droplet 182 while the looped arrangement storage locations 116 may include another type of large-volume droplet 180. For example, storage locations 116a, 116b, 116c, and 116d may include large-volume sample droplets 182a, 182b, 182c, and 182d, respectively. Further, storage locations 116e, 116f, 116g, 116h, 116i and 116j may include other large-volume droplets 180a, 180b, 180c, 180d, 180e and 182f, respectively.

During processing, any of one type of large-volume sample droplets 180 and/or another type of large-volume droplets 182 may be transported to mixing area 174. Mixing area 174 may comprise a distinct electrode layout and/or gap height gradient and may receive large-volume droplets 180 and/or another type of large-volume droplets 182 to be mixed prior to dispensing into the processing area 170. Mixing area 174 allows for mixing of larger volumes of liquid and may enable mixing of air sensitive reagents which may degenerate quickly. However, at any given time, the entire volume at any droplet may not be used completely during dispensing. Therefore, there may be reason to move any unused volume to waste area 172. Accordingly, electrode configuration 168 facilitates both the advantages of linear reservoir configuration 100 and looped reservoir configuration 160 and the ability to move any unused volume to waste.

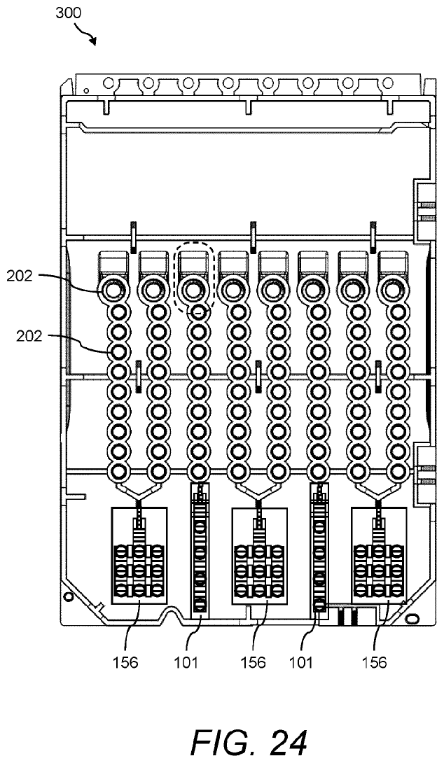

FIG. 24 is a plan view of an example of a microfluidics device 300 including the ganged reservoir configurations, in accordance with an embodiment of the disclosure. Microfluidics device 300 may be, for example, any type of droplet actuator, such as, but not limited to, a microfluidics device, a microfluidics cartridge, a DMF device, a DMF cartridge, a flow cell device, and the like. Microfluidics device 300 may include any arrangement of conventional wells or reservoirs 202 along with any arrangements of the ganged reservoir configurations. In this example, microfluidics device 300 may include arrangements of conventional wells or reservoirs 202 along with one or more 4×4-linear ganged reservoirs 101 and one or more ganged-reservoir array configurations 156.

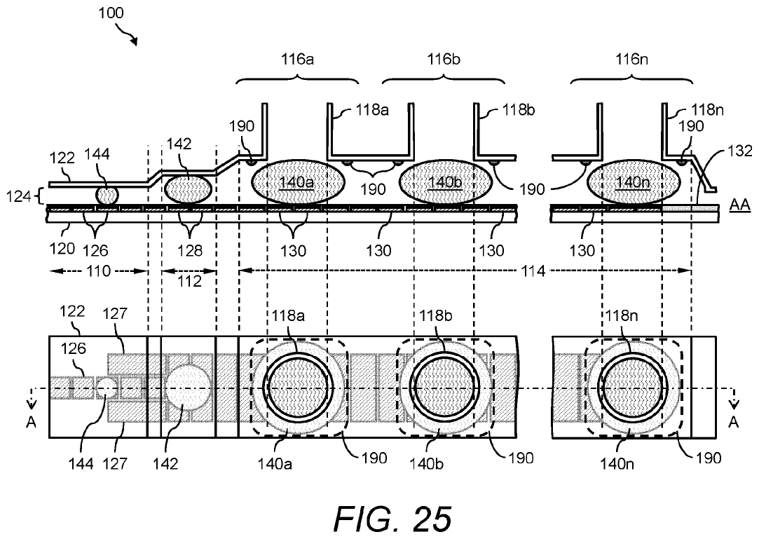

Referring now to FIG. 25 is a plan view and a cross-sectional view of linear ganged (or extended) reservoir configuration 100 that further includes a boundary feature 190, in accordance with an embodiment of the disclosure. To prevent large-volume droplets 140 from moving while no reservoir electrode 130 is activated, small structures (e.g., walls) may be provided can help to create a small barrier at each storage location 116. Accordingly, a boundary feature 190 may be included on top substrate 122 or bottom substrate 120 at each of the storage locations 116. The presence of boundary feature 190 requires some additional energy to move large-volume droplets 140 away from its storage location 116. This additional energy may be the actuation/activation of an adjacent electrode.

### Methods of Use

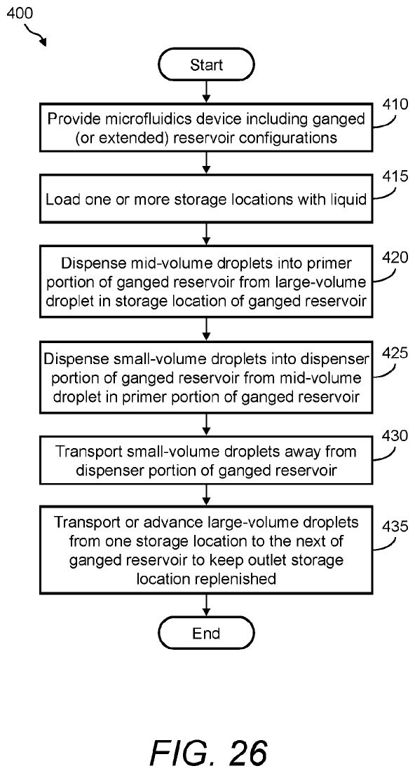

FIG. 26 is a flow diagram of method 400 of using the ganged reservoirs as described hereinabove with reference to FIG. 1 through FIG. 25, in accordance with an embodiment of the disclosure. Method 400 may include, but is not limited to, the following steps.

At step 410, a microfluidics device is provided that includes one or more ganged (or extended) reservoir configurations. For example, a microfluidics device is provided that may include one or more ganged (or extended) reservoir configurations, such as, but not limited to, any of the linear ganged reservoir configurations 100 (see FIG. 1, FIG. 2, FIG. 5, FIG. 6), 4×4-linear ganged reservoirs 101 (see FIG. 3, FIG. 4), 4×1-linear ganged reservoirs 102 (see FIG. 7, FIG. 8), any of the ganged reservoir configurations 150 (see FIG. 10), 4×4×4-ganged reservoirs 152 (see FIG. 11), any of the ganged-reservoir array configurations 154 (see FIG. 12), any of the ganged-reservoir array configurations 156 (see FIG. 13), 5×5×5 ganged-reservoir array 158 (see FIG. 14, FIG. 15), and looped ganged reservoir 160 (see FIG. 18, FIG. 19).

At step 415, one or more the storage locations are loaded with liquid. For example, the one or more storage locations may be loaded by a user using a micropipette. However, other means may be provided for loading the one or more storage locations including blister packs or reagent plates which interface with the DMF device. In one example, one or more storage locations 116 of linear ganged reservoir configuration 100 (see FIG. 2, FIG. 6) may be loaded with liquid via their respective loading ports 118. In another example, one or more storage locations 116 of 4×4-linear ganged reservoir 101 (see FIG. 4) may be loaded with liquid via their respective loading ports 118. In yet another example, one or more storage locations 116 of 4×1-linear ganged reservoir 102 (see FIG. 8) may be loaded with liquid via a singular loading port positioned distally from the primer portion 112 and large-volume droplets 140 may be moved via droplet operations to any storage location. In yet another example, one or more storage locations 116 of 5×5×5 ganged-reservoir array 158 (see FIG. 14, FIG. 15) may be loaded with liquid via their respective loading ports 118. In yet another example, one or more storage locations 116 of looped ganged reservoir 160 (see FIG. 18, FIG. 19) may be loaded with liquid via one or more loading ports. In yet another example, one or more storage locations 116 shown in electrode configuration 164 of FIG. 21 may be loaded with liquid. In still another example, one or more storage locations 116 shown in electrode configuration 166 of FIG. 22 may be loaded with liquid. In all cases, a large-volume droplet 140 may be formed in each storage location 116.

At step 420, mid-volume droplets are dispensed using droplet operations into the primer portion of the ganged reservoir from the large-volume droplet in the storage location of the ganged reservoir. For example, mid-volume droplets 142 may be dispensed into primer portion 112 of any of the ganged reservoirs from the large-volume droplet 140 in the outlet storage location 116 of the ganged reservoir. In one example, mid-volume droplets 142 may be dispensed into primer portion 112 of linear ganged reservoir configuration 100 (see FIG. 2, FIG. 6) from the large-volume droplet 140 in its outlet storage location 116. In another example, mid-volume droplets 142 may be dispensed into primer portion 112 of 4×4-linear ganged reservoir 101 (see FIG. 4) from the large-volume droplet 140 in its outlet storage location 116. In yet another example, mid-volume droplets 142 may be dispensed into primer portion 112 of 4×1-linear ganged reservoir 102 (see FIG. 8) from the large-volume droplet 140 in its outlet storage location 116.

At step 425, small-volume droplets are dispensed into the dispenser portion of the ganged reservoir from the mid-volume droplet in the primer portion of the ganged reservoir using droplet operations. For example, small-volume droplets 144 may be dispensed into dispenser portion 110 of any of the ganged reservoirs from the mid-volume droplet 142 in the outlet storage location 116 of the ganged reservoir. In one example, small-volume droplets 144 may be dispensed into dispenser portion 110 of linear ganged reservoir configuration 100 (see FIG. 2, FIG. 6) from the mid-volume droplet 142 in primer portion 112. In another example, small-volume droplets 144 may be dispensed into dispenser portion 110 of 4×4-linear ganged reservoir 101 (see FIG. 4) from the mid-volume droplet 142 in primer portion 112. In yet another example, small-volume droplets 144 may be dispensed into dispenser portion 110 of 4×1-linear ganged reservoir 102 (see FIG. 8) from the mid-volume droplet 142 in primer portion 112.

At step 430, the small-volume droplets may be transported away from the dispenser portion of the ganged reservoir for further processing. For example, small-volume droplets 144 may be transported away from dispenser portion 110 of any of the ganged reservoirs for further processing. In one example, small-volume droplets 144 may be transported away from dispenser portion 110 of linear ganged reservoir configuration 100 (see FIG. 2, FIG. 6). In another example, small-volume droplets 144 may be transported away from dispenser portion 110 of 4×4-linear ganged reservoir 101 (see FIG. 4). In yet another example, small-volume droplets 144 may be transported away from dispenser portion 110 of 4×1-linear ganged reservoir 102 (see FIG. 8).

At step 435, the large-volume droplets are transported or advanced from one storage location to the next of the ganged reservoir to keep the outlet storage location replenished. For example, in the ganged reservoir or reservoir configuration, large-volume droplets 140 may be transported or advanced from one storage location 116 to the next storage location 116 to keep the outlet storage location 116 replenished for dispensing mid-volume droplets 142 and then small-volume droplets 144. Further to the example, when the large-volume droplet 140 at the outlet storage location 116a is depleted, then the next large-volume droplet 140 may be transported (via droplet operations) into storage location 116a from storage location 116b. Further, all other upstream large-volume droplets 140 may be advanced toward the outlet storage location 116a.

Generally, using method 400 and, for example, linear ganged reservoir configuration 100, multiple large-volume droplets 140 at multiple storage locations 116 may be used to resupply mid-volume droplets 142 to the one primer portion 112 and then resupply small-volume droplets 144 to the one dispenser portion 110. Then, small-volume droplets 144 from dispenser portion 110 may be used to supply any processes of a microfluidics device (not shown).

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the invention shall also cover any such alternatives, modifications, variations or equivalents. It is intended that the following claims define the scope of the disclosure and that methods and structures within the scope of these claims and their equivalents be covered thereby.

1. A microfluidic cartridge comprising:
a top substrate;
a bottom substrate;
a gap between the top substrate and the bottom substrate; and
an electrode array disposed near the gap for performing droplet operations within the gap, the electrode array comprising,
one or more arrays of reservoir electrodes, each of the one or more arrays of reservoir electrodes configured to individually manipulate more than one large-volume droplets within the gap,
one or more arrays of priming electrodes for priming at least one of the more than one large-volume droplets, and
one or more arrays of dispensing electrodes for dispensing into the gap a small-volume droplet from a primed large-volume droplet.

2. (canceled)

3. (canceled)

4. The microfluidic device of claim 1, wherein the one or more arrays of reservoir electrodes and the one or more arrays of priming electrodes are the same.

5. The microfluidic device of claim 1, wherein the electrode array further comprises more than one array of reservoir electrodes.

6. The microfluidic device of claim 5, wherein the electrode array further comprises one or more arrays of bridging electrodes between each of the more than one array of reservoir electrodes for manipulating any one of the more than one large-volume droplets between each of the more than one array of reservoir electrodes.

7. The microfluidic device of claim 1, wherein at least one of the one or more arrays of reservoir electrodes is in a linear configuration.

8. The microfluidic device of claim 1, wherein at least one of the one or more arrays of reservoir electrodes is in a loop configuration.

9. The microfluidic device of claim 1, wherein a gap height of the gap is from about 50 pm to about 3000 pm.

10. The microfluidic device of claim 1, wherein a gap height of the gap is greater near the one or more arrays of reservoir electrodes when compared with the gap height of the gap near the one or more arrays of dispensing electrodes.

11. The microfluidic device of claim 9, wherein the gap height of the gap near the one or more arrays of reservoir electrodes is from about 300 pm to about 3000 pm.

12. The microfluidic device of claim 9, wherein the gap height of the gap near the one or more arrays of dispensing electrodes is from about 50 pm to about 500 pm.

13. The microfluidic device of claim 1, wherein the one or more large-volume droplets has a volume from about 20 pL to about 5000 pL.

14. The microfluidic device of claim 1, wherein the small-volume droplet has a volume from about 1 pL to about 10 pL.

15. The microfluidic device of claim 1, wherein the top plate comprises one or more loading ports for providing the more than one large-volume droplets.

16. The microfluidic device of claim 15, wherein the one or more loading ports is configured to interface with a micropipette.

17. The microfluidic device of claim 1, wherein the electrode array further comprises an array of processing electrodes for manipulating the small-volume droplet.

18. The microfluidic device of claim 17, wherein manipulating the small-volume droplet comprises either
i) mixing the small-volume droplet with one or more additional droplets;
ii) splitting the small-volume droplet into one or more additional droplets; or
iii) sensing one or more reagents, analytes, or samples of the small-volume droplet.

19. The microfluidic device of claim 1, wherein the electrode array further comprises an array of waste electrodes for disposing of an unused portion of any one of the more than one large-volume droplets.

20. The microfluidic device of claim 1, wherein the electrode array further comprises an array of mixing electrodes for mixing two or more large-volume droplets.

21. The microfluidic device of claim 1, wherein at least one of the top plate or bottom plate further comprises a boundary feature to prevent migration of the more than one large-volume droplets.

22. The microfluidic device of claim 21, wherein the boundary feature comprises a protrusion disposed on either the top substrate or bottom substrate.

23.-46. (canceled)
